(19) United States
(12) Patent Application Publication
(10) Pub. No.: US 2022/0401411 A1
(43) Pub. Date: Dec. 22, 2022

(54) ZAFIRLUKAST DERIVATIVES FOR USE AS CONTRACEPTIVE AGENTS

(71) Applicant: Rigshospitalet, Copenhagen Ø (DK)

(72) Inventor: Anders REHFELD, Copenhagen Ø (DK)

(21) Appl. No.: 17/763,694

(22) PCT Filed: Oct. 8, 2020
(86) PCT No.: PCT/EP2020/078205

§ 371 Date: Mar. 25, 2022

(30) Foreign Application Priority Data
Oct. 8, 2019 (DK) PA 2019 01185

(51) Int. Cl. A61K 31/404; A61P 15/16; A61K 31/56

(52) U.S. Cl. CPC A61K 31/404; A61P 15/16; A61K 31/56

(57) ABSTRACT

The present invention concerns compounds and their use in contraception. These compounds are derivatives of zafirlukast and have been found to inhibit CatSper in human sperm cells in low concentrations.

### FIELD OF THE INVENTION

The present invention concerns compounds and their use in contraception. These compounds are derivatives of zafirlukast of formula I as defined below. The contraceptive compounds disclosed herein may be systemic or intravaginal applied in the form of a foam, cream or gel, or in unit form of a pill, vaginal contraceptive film (VCF), suppository, sponge, transdermal or hypodermal patches or a slow release intravaginal device or intrauterine device (IUD) such as a drug-impregnated silicone elastomer vaginal ring or polymeric IUD.

### BACKGROUND OF THE INVENTION

For a human sperm cell to be able to successfully and naturally fertilize the human egg, the functions of the sperm cell must be precisely regulated during its journey through the female reproductive tract. Many sperm cell functions are controlled via the intracellular Ca²⁺-concentration, e.g., sperm motility, chemotaxis, and acrosome reaction. Without being bound to a particular theory, channel-mediated Ca²⁺-influx in human sperm cells is believed almost exclusively to occur via CatSper (Cationic channel of Sperm), which is only expressed in sperm cells. Human CatSper is activated by the female sex hormone progesterone and prostaglandins, both released in high amounts from the cumulus cells that surround the egg. The activation of CatSper by these ligands leads to a rapid Ca²⁺-influx into the sperm cell. The progesterone-induced Ca²⁺-influx in the sperm cells mediates chemotaxis towards the egg, controls sperm motility and stimulates the acrosome reaction. Triggering of these individual Ca²⁺-controlled sperm functions at the correct time and in the correct order is crucial for fertilization of the egg. In line with this, a suboptimal progesterone-induced Ca²⁺-influx has been found to be associated with reduced male fertility and functional CatSper is believed to be absolutely required for male fertility.

As CatSper is exclusively expressed in sperm cells, it represents a promising specific and safe target for novel male contraceptives. As millions of new sperm cells are constantly produced through spermatogenesis, compounds targeting CatSper would also work in a fully reversible fashion, even if they bind irreversibly. Presently, the best inhibitors of human CatSper are RU1968, Lupeol, and Pristimerin, although the inhibitory effect of Lupeol and Pristimerin could not be reproduced in several recent publications and is thus debated. RU1968, Lupeol and Pristimerin are all steroid-like; RU1968 is synthesized from a (±) estrone methyl ether steroid backbone, and both Lupeol and Pristimerin are steroid-like triterpenoids (precursors of all steroid hormones in animals). RU1968, Lupeol and Pristimerin could therefore exert endocrine side-effects if used as contraceptives. A non-steroidal inhibitor of human CatSper is thus much needed.

### SUMMARY OF THE INVENTION

In one aspect, the present invention concerns the use of a compound of formula I or a pharmaceutically acceptable salt thereof:

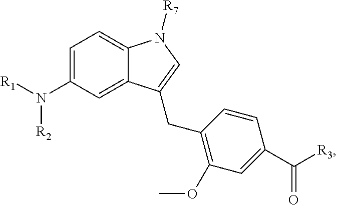

wherein

R₁ and R₂ are independently selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S, R₁₂OCO—, R₁₂CO—, and R₁₂NCO;

R₁₂ is selected from the group consisting of optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S;

R₃ is selected from the group consisting of OR₄ and NR₅R₆;

R₄ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, and an optionally substituted 3- to 8-membered heterocyclyl containing one or more heteroatoms selected from the group consisting of N, O, and S;

R₅ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S, and R₅₂SO₂—;

R₅₂ is selected from the group consisting of optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S;

R₆ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, and an optionally substituted 3- to 8-membered heterocyclyl containing one or more heteroatoms selected from the group consisting of N, O, and S; and

R₇ is a straight or branched C₁₋₈ alkyl, wherein 1 to 2 carbon are optionally replaced by nitrogen, preferably methyl;

as a contraceptive.

In another aspect, the present invention concerns a method of contraception including the step of administering a compound of formula I to a subject in need thereof. In a further aspect, the present invention concerns a compound of formula I or a pharmaceutically acceptable salt thereof, with the proviso that said compound of formula I is not zafirlukast.

### BRIEF DESCRIPTION OF THE DRAWINGS

FIGS. 1-4 demonstrate the inhibition of CatSper in human sperm cells by zafirlukast. Isolated live and motile human sperm cells were loaded with a Ca²⁺-sensitive fluorophore, washed and aliquoted to a 384-well plate placed in a fluorescence plate reader. Readouts in ΔF/F₀ (%) on the y-axis reflect the intracellular concentration of Ca²⁺ ([Ca²⁺],) in the sperm cells as a function of time (FIG. 1, 2, 4) or dose of zafirlukast (FIG. 3).

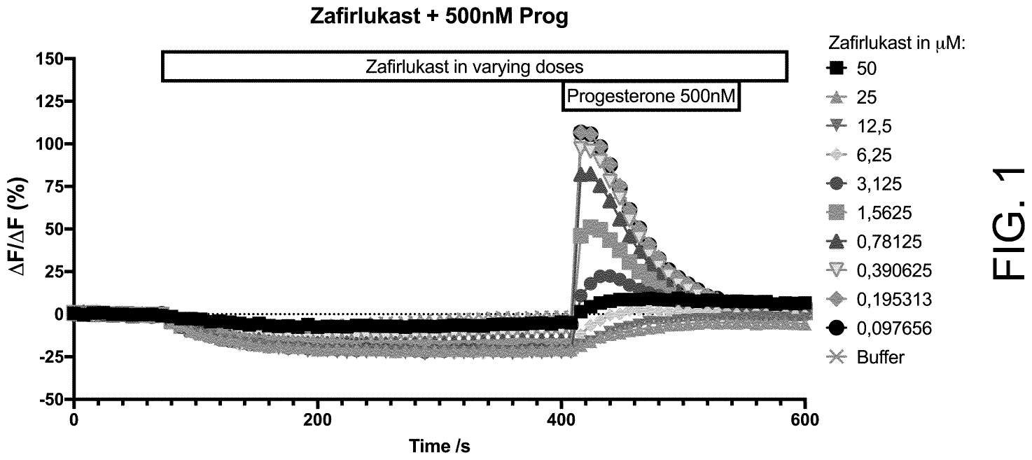

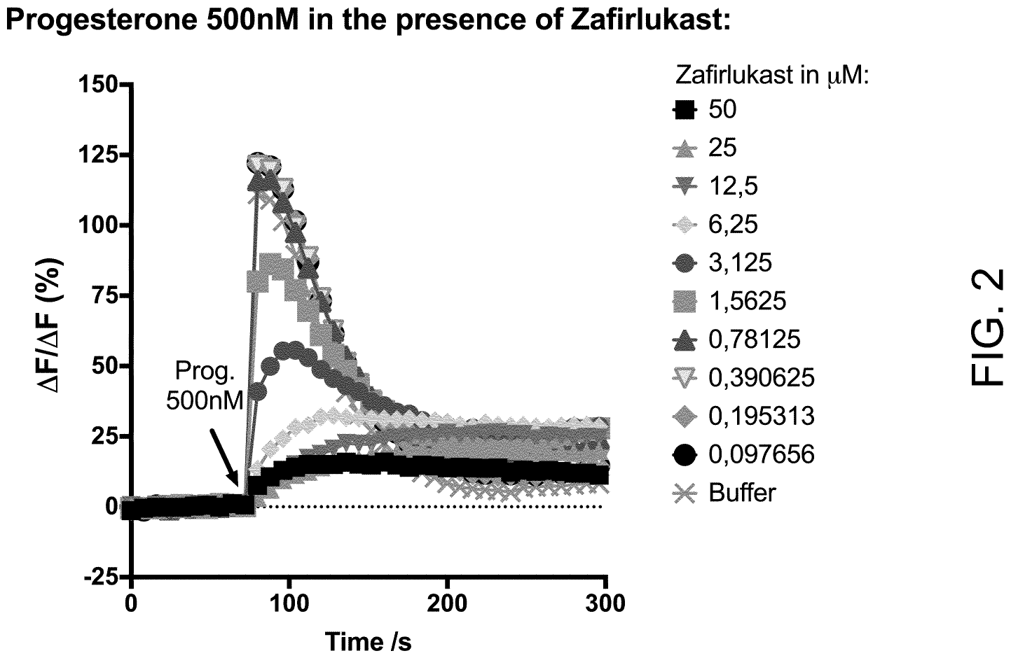

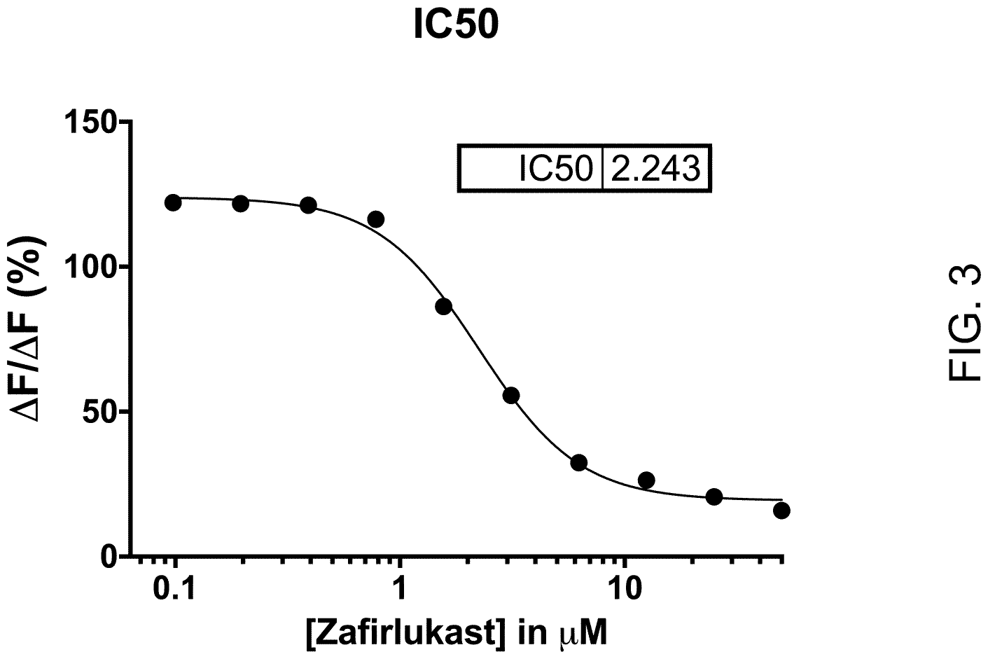

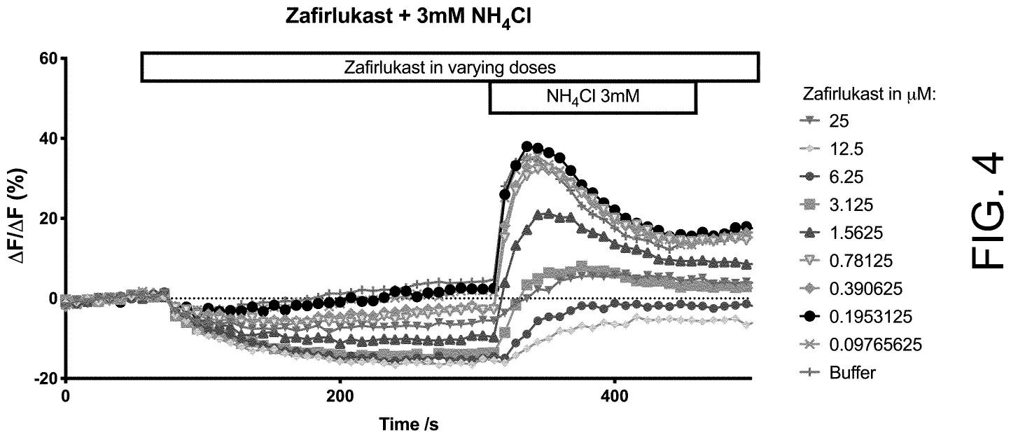

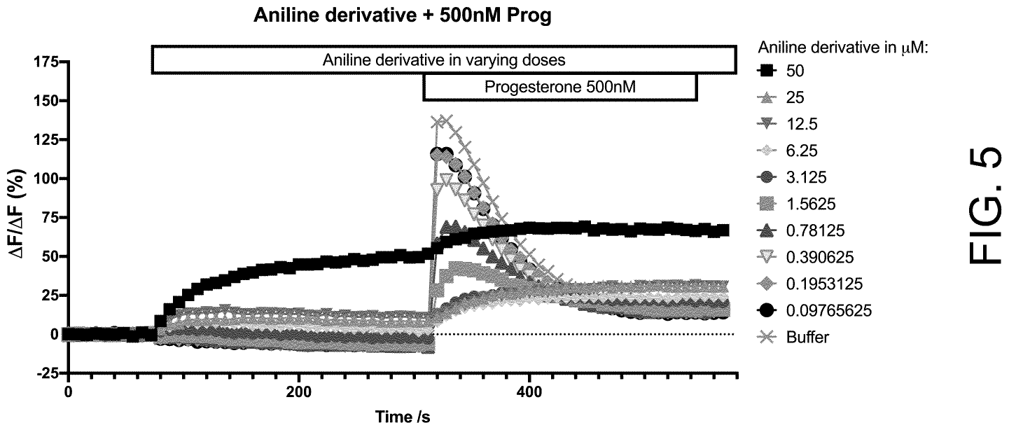

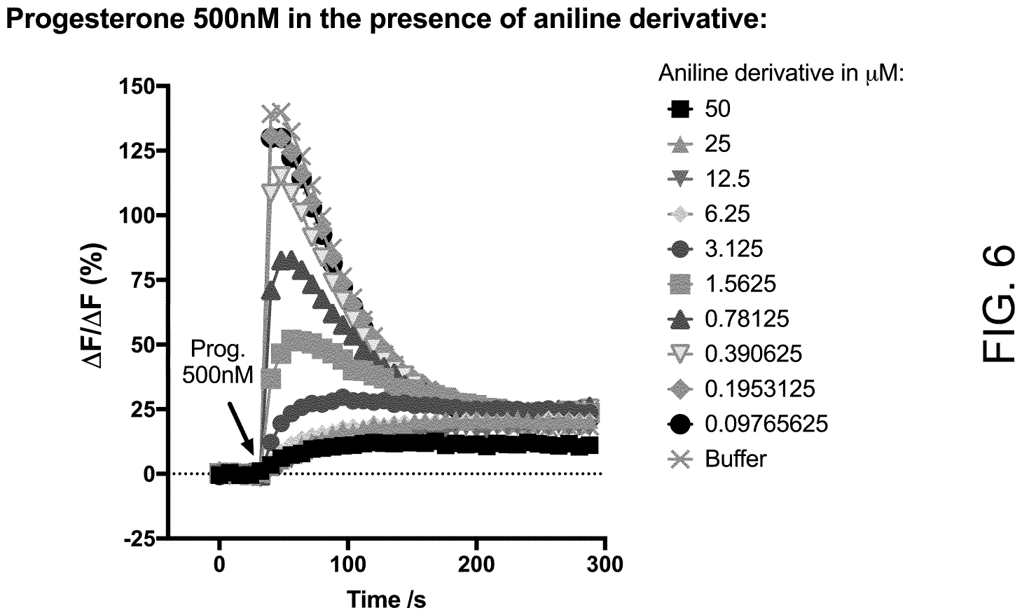

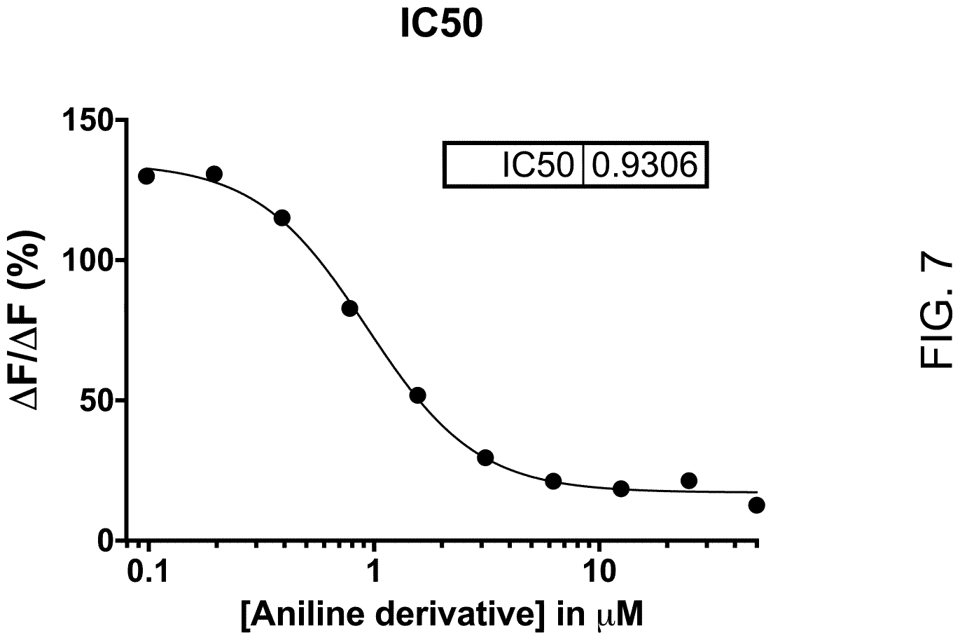

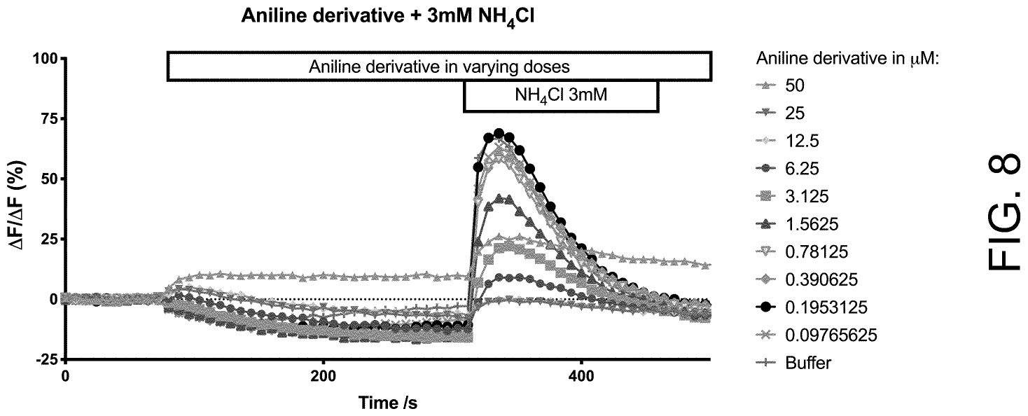

After measuring the baseline fluorescence for 80s serially diluted doses of zafirlukast as well as a negative buffer control (FIGS. 1 and 4) were added to the sperm cells. Hereafter the measurement was continued for a few minutes before a second addition of 500 nM progesterone (FIG. 1) or 3 mM NH₄Cl (FIG. 4) to activate CatSper. As can be seen, zafirlukast in low μM doses inhibits the activation of human CatSper by both progesterone (FIG. 1) and intracellular alkalization via NH₄Cl (FIG. 4), resembling the phenotype found for human sperm cells lacking functional CatSper. CatSper-signals induced by 500 nM progesterone (FIG. 2) in the presence of serially diluted doses of zafirlukast are used for the dose inhibition curve for zafirlukast on progesterone-induced signals through CatSper (FIG. 3).

FIGS. 5-8 demonstrate the inhibition of CatSper in human sperm cells by the aniline derivative. Isolated live and motile human sperm cells were loaded with a Ca²⁺-sensitive fluorophore, washed and aliquoted to a 384-well plate placed in a fluorescence plate reader. Readouts in ΔF/F₀ (%) on the y-axis reflect the intracellular concentration of Ca²⁺ ([Ca²⁺],) in the sperm cells as a function of time (FIG. 5, 6, 8) or dose of aniline derivate (FIG. 7).

After measuring the baseline fluorescence for 80s serially diluted doses of aniline derivative as well as a negative buffer control (FIGS. 5 and 8) were added to the sperm cells. Hereafter the measurement was continued for a few minutes before a second addition of 500 nM progesterone (FIG. 5) or 3 mM NH₄Cl (FIG. 8) to activate CatSper. As can be seen, aniline derivative in low μM doses inhibits the activation of human CatSper by both progesterone (FIG. 5) and intracellular alkalization via NH₄Cl (FIG. 8), resembling the phenotype found for human sperm cells lacking functional CatSper. CatSper-signals induced by 500 nM progesterone (FIG. 6) in the presence of serially diluted doses of the aniline derivative are used for the dose inhibition curve for aniline derivative on progesterone-induced signals through CatSper (FIG. 7).

### DETAILED DESCRIPTION OF THE INVENTION

### Definitions

In the present context, the term “C₁₋₄ alkyl” is intended to mean a linear or branched hydrocarbon group having 1 to 4 carbon atoms, such as methyl, ethyl, n-propyl, iso-propyl, n-butyl, iso-butyl, sec-butyl, and tert-butyl.

Similarly, the term “C₂₋₄ alkenyl” is intended to cover linear or branched hydrocarbon groups having 2 to 4 carbon atoms and comprising a double bond. Examples of C₂₋₄ alkenyl groups are vinyl, allyl, and butenyl. Preferred examples of alkenyl are vinyl and allyl, especially allyl.

In the present context the term “C₂₋₄ alkynyl” is intended to mean a linear or branched hydrocarbon group having 2 to 4 carbon atoms and containing a triple bond. Illustrative examples of C₂₋₄ alkynyl groups include acetylene, propynyl, butynyl, as well as branched forms of these. The position of unsaturation (the triple bond) may be at any position along the carbon chain. More than one bond may be unsaturated such that the “C₂₋₄ alkynyl” is a di-yne as is known to the person skilled in the art.

Herein, the term “halogen” includes fluoro, chloro, bromo, and iodo, more particularly, fluoro, chloro and bromo.

In the present context the term “aromatic ring or ring system” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl.

In the present context, the term “heterocyclic ring or ring system” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclic groups are imidazolidine, piperazine, hexahydropyridazine, hexahydropyrimidine, diazepane, diazocane, pyrrolidine, piperidine, azepane, azocane, aziridine, azirine, azetidine, pyroline, tropane, oxazinane (morpholine), azepine, dihydroazepine, tetrahydroazepine, hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane and oxathiepane.

In the present context, the term “optionally substituted” is intended to mean that the group in question may be substituted at least once. Furthermore, the term “optionally substituted” may also mean that the group in question is unsubstituted.

The compounds of the present invention can be in a free form or in the form of a pharmaceutically acceptable salt. In the context of the present invention, the term “pharmaceutically acceptable salt” is to be understood as a salt formed with either a base or an acid, wherein the resulting counter-ion does not significantly add to the toxicity of the compound of the present invention.

Examples of pharmaceutically acceptable salts include inorganic acid salts such as hydrochloride, sulfate, nitrate, phosphate or hydrobromide, etc., organic acid salts such as acetate, fumarate, oxalate, citrate, methanesulfonate, benzenesulfonate, p-toluenesulfonate or maleate, etc. Also, when the compound has a substituent such as carboxyl group, there may be mentioned a salt with a base (for example, alkali metal salt such as sodium salt, potassium salt, etc. or alkaline earth metal salt such as calcium salt, etc.).

Contraceptive Use

In one aspect, the present invention concerns the use of a compound of formula I or a pharmaceutically acceptable salt thereof:

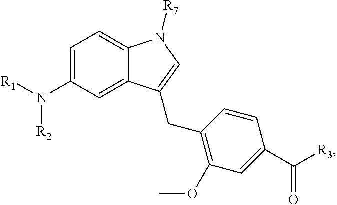

wherein

R₁ and R₂ are independently selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S, R₁₂OCO—, R₁₂CO—, and R₁₂NCO;

R₁₂ is selected from the group consisting of optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S;

R₃ is selected from the group consisting of OR₄ and NR₅R₆;

R₄ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, and an optionally substituted 3- to 8-membered heterocyclyl containing one or more heteroatoms selected from the group consisting of N, O, and S;

R₅ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S, and R₅₂SO₂—;

R₅₂ is selected from the group consisting of optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S;

R₆ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, and an optionally substituted 3- to 8-membered heterocyclyl containing one or more heteroatoms selected from the group consisting of N, O, and S; and

R₇ is a straight or branched C₁₋₈ alkyl, wherein 1 to 2 carbon are optionally replaced by nitrogen, preferably methyl;

as a contraceptive.

In one embodiment, R₇ contains two nitrogen atoms in the chain. In a further embodiment, R₇ is:

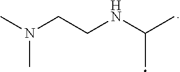

In still a further embodiment, R₇ is methyl.

The contraceptive compounds described above can be used as a hormone-free or non-hormonal method of birth control.

In one embodiment, the compound of formula I is a compound of formula Ia:

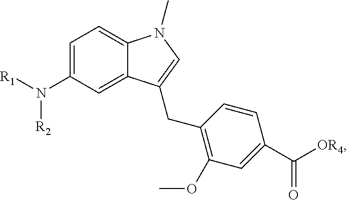

wherein R₁, R₂, and R₄ are as defined for formula I.

In another embodiment, the compound of formula I is a compound of formula Ib:

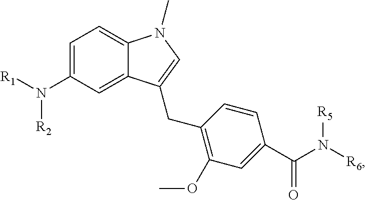

wherein R₁, R₂, R₅, R₅₂ and R₆ are as defined for formula I.

In still another embodiment, the compound of formula I is a compound of formula II:

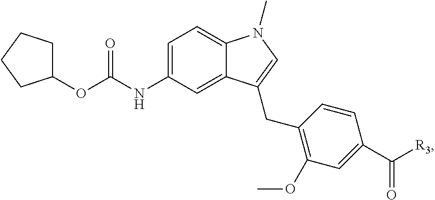

wherein R₃, R₄, R₅, R₅₂ and R₆ are as defined for formula I.

In yet another embodiment, the compound of formula I is a compound of formula III:

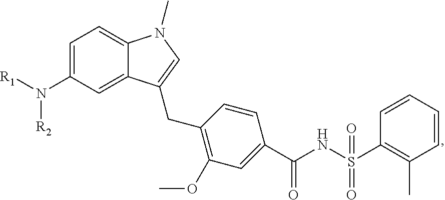

wherein R₁ and R₂ are as defined for formula I.

In a further embodiment, R₁ and R₂ are independently selected from hydrogen, optionally substituted C₁₋₄ alkyl, and R₁₂OCO—, wherein R₁₂ is selected from optionally substituted C₁₋₄ alkyl and optionally substituted 3- to 8-membered cyclyl. In still a further embodiment, R₁ and R₂ are both hydrogen. In yet a further embodiment, R₃ is OR₄ and R₄ is selected from the group consisting of hydrogen, optionally substituted C₁₋₄ alkyl, optionally substituted 3- to 8-membered cyclyl, and an optionally substituted 6- to 10-membered aromatic ring or ring system. In still a further embodiment, R₃ is NR₅R₆ and R₅ is selected from the group consisting of hydrogen, optionally substituted C₁₋₄ alkyl, optionally substituted 3- to 8-membered cyclyl, an optionally substituted 6- to 10-membered aromatic ring or ring system, and R₅₂SO₂—, wherein R₅₂ is selected from the group consisting of optionally substituted C₁₋₄ alkyl, optionally substituted 3- to 8-membered cyclic, and an optionally substituted 6- to 10-membered aromatic ring or ring system, and R_{B} is hydrogen. In yet a further embodiment, the compound of formula I is zafirlukast.

Compounds

The derivatives of zafirlukast of formula I as defined herein are, except for zafirlukast, novel compounds. Therefore, in another aspect of the present invention, it concerns a compound of formula I, Ia, Ib, II, or III as defined herein or a pharmaceutically acceptable salt thereof, with the proviso that said compound is not zafirlukast.

The compounds of the invention may be prepared from zafirlukast as the starting point. In one embodiment, compounds of formula III are prepared from zafirlukast by acid-mediated carbamate deprotection that are well known in the art (such as the addition of e.g. trifluoroacetic acid) and subsequent functionalization of the resulting amine nitrogen (such as through substitution reactions well known in the art, e.g. by nucleophilic substitution with alkyl halides). In another embodiment, compounds of formula II are prepared from zafirlukast by reductive sulfonamide deprotection using reagents well known in the art (such as alkali metals (Li, Na, K), lithium naphthalenide, SmI₂ with HMPA, or LiAlH₄ in the presence of nickel compounds) and subsequent amide functionalization (in the case where R₃ is NR₅R₆) or by reductive sulfonamide deprotection, amide hydrolysis (alkaline or acidic), and subsequent acid functionalization. Another possibility is direct functionalization of the acidic sulfonamide proton. A combination of these synthetic strategies can be used for all compounds of formula I. Methods of preparing zafirlukast and derivatives thereof may further be found in EP 0 199 543.

Pharmaceutical Formulation

The compounds of the present invention are intended for use as a medicament. The compounds of the invention may in principle be applied on their own, but they are preferably formulated with a pharmaceutically acceptable carrier. A pharmaceutically acceptable carrier is an inert carrier suitable for each administration method and can be formulated into conventional pharmaceutical preparation (tablets, granules, capsules, powder, solution, suspension, emulsion, injection, infusion, etc.). As such a carrier there may be mentioned, for example, a binder, an excipient, a lubricant, a disintegrant and the like, which are pharmaceutically acceptable. When they are used as an injection solution or an infusion solution, they can be formulated by using distilled water for injection, physiological saline, an aqueous glucose solution.

Administration

The administration method of the compounds of the present invention is not particularly limited, and a usual oral or parenteral administration method (intravenous, intramuscular, subcutaneous, percutaneous, intranasal, transmucosal, enteral, intravaginal, etc.) can be applied.

In one or more exemplary embodiments, the contraceptive compounds are in the form of a pill, patch, microneedle or intravaginal form such as film, foam, cream, or gel, particularly in a predetermined, unit dosage effective for contraception.

In one or more exemplary embodiments, the contraceptive compounds are in unit form of a vaginal contraceptive film (VCF), suppository, sponge, or slow release intravaginal devices or intrauterine devices (IUDs) such as drug-impregnated silicone elastomer vaginal rings or polymeric IUDs.

Dosage

The dosage of the zafirlukast derivatives or a pharmaceutically acceptable salt thereof of the present invention may optionally be set in a range of an effective amount sufficient for showing a pharmacological effect, in accordance with the potency or characteristics of the compound to be used as an effective ingredient. The dosage may vary depending on administration method, age, body weight or conditions of a patient.

Species

The compounds of formula I as defined herein may be used for contraception in any species with CatSper expressed in the male gametes.

In one or more exemplary embodiments, the compound of formula I is used in human males and females.

In one or more exemplary embodiments, the compound of formula I is used in human females.

In one or more exemplary embodiments, the compound of formula I is used in human males.

Reversible Mode of Action

Compounds targeting CatSper would work in a fully reversible fashion, as new sperm cells are constantly produced through spermatogenesis. Thus, the compounds described herein may act as reversible, non-hormonal human male contraceptives. Use of compounds targeting CatSper should therefore not affect the future fertility potential of the users, unlike, e.g., vasectomy.

Combination with Triterpenoid Compounds

The compounds according to the present invention may be used in contraception in combination with other compounds known to have an effect on CatSper. Such compounds include triterpenoid compounds as disclosed in WO 2018/128956. Hence, in one embodiment, the present invention concerns the use of a compound of formula I as defined herein in combination with a triterpenoid as a contraceptive. In another embodiment, the present invention concerns the use of a compound of formula I in combination with pristimerin, lupeol, and/or celastrol. In still another embodiment, the present invention concerns the use of a compound of formula Ia in combination with pristimerin, lupeol, and/or celastrol. In yet another embodiment, the present invention concerns the use of a compound of formula Ib in combination with pristimerin, lupeol, and/or celastrol. In still a further embodiment, the present invention concerns the use of a compound of formula II in combination with pristimerin, lupeol, and/or celastrol. In yet a further embodiment, the present invention concerns the use of a compound of formula III in combination with pristimerin, lupeol, and/or celastrol.

### EXAMPLES

### Example 1—Inhibition of CatSper by Zafirlukast

Important sperm cell functions are controlled via the intracellular Ca²⁺-concentration ([Ca²⁺]), e.g., sperm motility, chemotaxis, and acrosome reaction. Interestingly, channel-mediated Ca²⁺-influx in human sperm cells seems almost exclusively to occur via CatSper, which is only expressed in sperm cells.

Human CatSper is activated by the female sex hormone progesterone and prostaglandins, both released in high amounts from the cumulus cells that surround the egg. The activation of CatSper by these ligands leads to a rapid Ca²⁺-influx into the sperm cell. Additionally, CatSper can be activated by intracellular alkalization, e.g. by addition of NH₄Cl to the sperm cell medium. The progesterone-induced Ca²⁺-influx in the sperm cells mediates chemotaxis towards the egg, controls sperm motility and stimulates the acrosome reaction. Triggering of these individual [Ca²⁺]-controlled sperm functions at the correct time and in the correct order is crucial for fertilization of the egg. In line with this, a suboptimal progesterone-induced Ca²⁺-influx has been found to be associated with reduced male fertility and functional CatSper is absolutely required for male fertility, i.e. human sperm cells lacking functional CatSper cannot fertilize the egg naturally.

To assess the effect of zafirlukast on CatSper in human sperm cells, we first isolated motile sperm cells from freshly ejaculated semen samples of healthy human donors through the swim-up method. Then we loaded these sperm cells with a Ca²⁺-fluorophore, i.e. a molecule that emits light more intensively when [Ca²⁺], increases. Hereafter we aliquoted these fluorophore-loaded sperm cells to the wells of micro-well plates and inserted the plate into a fluorescence plate reader, that measures the light emitted from the Ca²⁺-fluorophore. After measuring the baseline fluorescence for 80s we added to the wells serially diluted doses of zafirlukast (FIGS. 1 and 4). Hereafter we continued the measurement for a few minutes before a second addition of 500 nM progesterone (FIG. 1) or 3 mM NH₄Cl (FIG. 4) to activate CatSper. As can be seen, zafirlukast in low μM doses inhibits the activation of human CatSper by both progesterone (FIG. 1) and intracellular alkalization via NH₄Cl (FIG. 4), resembling the phenotype found for human sperm cells lacking functional CatSper.

An IC₅₀ value of approximately 2.2 μM was determined for zafirlukast (FIG. 2-3).

### Example 2—Inhibition of CatSper by the Aniline Derivative

The aniline derivative of zafirlukast has the following formula:

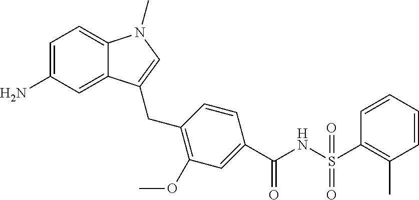 [SMILES: COc1cc(C(=O)NS(=O)(=O)c2ccccc2C)ccc1Cc1cn(C)c2ccc(N)cc12]

The aniline derivative was prepared by dissolving zafirlukast in methanol with stirring. Acyl chloride was added to the mixture with stirring and was left to react for 48 hours.

To assess the effect of the aniline derivative on CatSper in human sperm cells, we first isolated motile sperm cells from freshly ejaculated semen samples of healthy human donors through the swim-up method. Then we loaded these sperm cells with a Ca²⁺-fluorophore, i.e. a molecule that emits light more intensively when [Ca²*], increases. Hereafter we aliquoted these fluorophore-loaded sperm cells to the wells of micro-well plates and inserted the plate into a fluorescence plate reader, that measures the light emitted from the Ca²⁺-fluorophore. After measuring the baseline fluorescence for 80s we added to the wells serially diluted doses of the aniline derivative (FIGS. 5 and 8). Hereafter we continued the measurement for a few minutes before a second addition of 500 nM progesterone (FIG. 5) or 3 mM NH₄Cl (FIG. 8) to activate CatSper. As can be seen, the aniline derivative in low μM doses inhibits the activation of human CatSper by both progesterone (FIG. 5) and intracellular alkalization via NH₄Cl (FIG. 8), resembling the phenotype found for human sperm cells lacking functional CatSper.

An IC₅₀ value of approximately 0.9 μM was determined for the aniline derivative (FIG. 6-7).

1. A method of contraception comprising:
administering a compound of formula I or a pharmaceutically acceptable salt thereof: 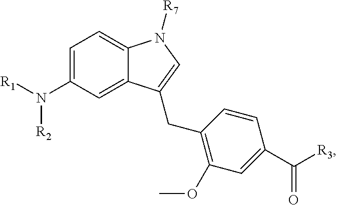
Wherein:
R₁ and R₂ are independently selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S, R₁₂OCO—, R₁₂CO—, and R₁₂NCO;
R₁₂ is selected from the group consisting of optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S;
R₃ is selected from the group consisting of OR₄ and NR₅R₆;
R₄ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, and an optionally substituted 3- to 8-membered heterocyclyl containing one or more heteroatoms selected from the group consisting of N, O, and S;
R₅ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S, and R₅₂SO₂—;
R₅₂ is selected from the group consisting of optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S;
R₆ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclic, an optionally substituted 6- to 10-membered aromatic ring or ring system, and an optionally substituted 3- to 8-membered heterocyclyl containing one or more heteroatoms selected from the group consisting of N, O, and S; and
R₇ is a straight or branched C₁₋₈ alkyl, wherein 1 to 2 carbon are optionally replaced by nitrogen, preferably methyl; and
wherein the administering comprises administering in human males.

2. The method according to claim 1, wherein the compound of formula I is a compound of formula Ia: 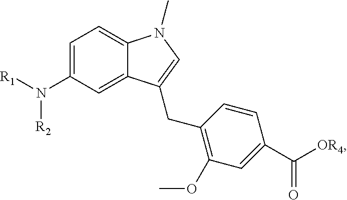
wherein R₁, R₂, and R₄ are as defined in claim 1.

3. The method according to claim 1, wherein the compound of formula I is a compound of formula Ib: 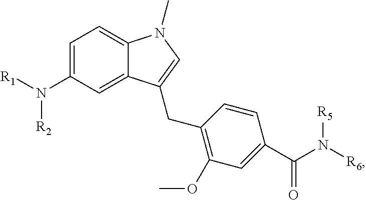
wherein R₁, R₂, R₅, R₅₂ and R₆ are as defined in claim 1.

4. The method according to claim 1, wherein the compound of formula I is a compound of formula II: 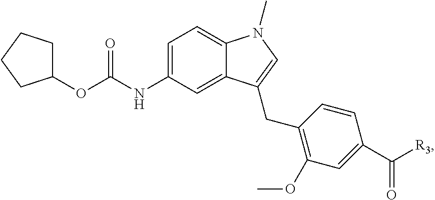
wherein R₃, R₄, R₅, R₅₂ and R₆ are as defined in claim 1.

5. The method according to claim 1, wherein the compound of formula I is a compound of formula III: 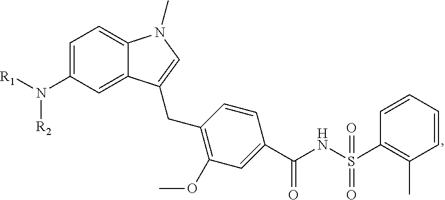
wherein R₁ and R₂ are as defined in claim 1.

6. The method according to claim 1, wherein the compound of formula I is zafirlukast.

7. The method according to claim 1, wherein the compound of formula I is combined with a triterpenoid.

8. The method according to claim 7 wherein the compound of formula I is combined with pristimerin, lupeol, and/or celastrol.

9. A compound as defined in claim 1, with the proviso that said compound is not zafirlukast.

10. (canceled)

11. The method according to claim 1, wherein the compound of formula I is used in species with CatSper expressed in the male gametes.

12. The method according to claim 1, wherein the administering comprises administering orally.

13. The method according to claim 1, wherein the administering comprises administering via a parenteral administration method.

14. A method of contraception comprising:
administering a compound of formula I or a pharmaceutically acceptable salt thereof: 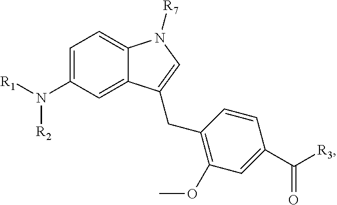
Wherein:
R₁ and R₂ are independently selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S, R₁₂OCO—, R₁₂CO—, and R₁₂NCO;
R₁₂ is selected from the group consisting of optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S;
R₃ is selected from the group consisting of OR₄ and NR₅R₆;
R₄ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, and an optionally substituted 3- to 8-membered heterocyclyl containing one or more heteroatoms selected from the group consisting of N, O, and S;
R₅ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S, and R₅₂SO₂—;
R₅₂ is selected from the group consisting of optionally substituted C₁₋₄ alkyl, optionally substituted C₂₋₄ alkenyl, optionally substituted C₂₋₄ alkynyl, optionally substituted 3- to 8-membered cyclyl, an optionally substituted 6- to 10-membered aromatic ring or ring system, an optionally substituted 3- to 8-membered heterocyclyl containing one or more heteroatoms selected from the group consisting of N, O, and S;
R₆ is selected from the group consisting of hydrogen, optionally substituted C₁₋₄ alkyl, optionally substituted C₂₋₄ alkenyl, optionally substituted C₂₋₄ alkynyl, optionally substituted 3- to 8-membered cyclic, an optionally substituted 6- to 10-membered aromatic ring or ring system, and an optionally substituted 3- to 8-membered heterocyclyl containing one or more heteroatoms selected from the group consisting of N, O, and S; and
R₇ is a straight or branched C₁₋₈ alkyl, wherein 1 to 2 carbon are optionally replaced by nitrogen, preferably methyl.
